(19) United States
(12) Patent Application Publication
(10) Pub. No.: US 2012/0035615 A1
(43) Pub. Date: Feb. 9, 2012

(54) Composite Stylet

(75) Inventors: Kurt J. Koester, Valencia, CA (US); Chuladatta Thenuwara, Castaic, CA (US); Timothy Beerling, Los Angeles, CA (US)

(73) Assignee: ADVANCED BIONICS, LLC, Valencia, CA (US)

(21) Appl. No.: 12/470,990

(22) Filed: May 22, 2009

(51) Int. Cl. A61B 17/00; A61N 1/05

(52) U.S. Cl. 606/129; 607/137

(57) ABSTRACT

A composite stylet for facilitating insertion of an implantable electrode array into either a left or right cochlea comprises a composite having a glass transition temperature between room temperature and body temperature. While relatively stiff and straight at room temperature, the composite stylet is slidably inserted into a longitudinal lumen of the electrode array. The electrode array is then inserted into the cochlea. As the composite stylet within the electrode array warms to body temperature, it becomes compliant, allowing the electrode array to assume a spiral shape. The proximal end of the composite stylet, which is not inserted into the body, retains its stiffness to aid the implanter in inserting the electrode array.

### FIELD OF INVENTION

The present invention relates to implantable stimulation devices, e.g., cochlear prostheses used to electrically stimulate the auditory nerve, and more particularly to a stylet for insertion of an electrode array into the cochlea.

### BACKGROUND OF INVENTION

Hearing loss, which may be due to many different causes, is generally of two types: conductive and sensorineural. Of these, conductive hearing loss occurs where the normal mechanical pathways for sound to reach the hair cells in the cochlea are impeded, for example, by damage to the ossicles. Conductive hearing loss may often be helped by use of conventional hearing aids, which amplify sound so that acoustic information does reach the cochlea and the hair cells. Some types of conductive hearing loss are also treatable by surgical procedures.

In many people who are profoundly deaf, however, the reason for their deafness is sensorineural hearing loss. This type of hearing loss is due to the absence or the destruction of the hair cells in the cochlea which are needed to transduce acoustic signals into auditory nerve impulses. These people may be unable to derive significant benefits from conventional hearing aid systems alone, no matter how loud the acoustic stimulus is made, because their mechanisms for transducing sound energy into auditory nerve impulses have been damaged.

To overcome sensorineural deafness, there have been developed numerous cochlear implant systems, or cochlear prostheses, which seek to bypass the hair cells in the vicinity of the radially outer wall of the cochlea by presenting electrical stimulation to the auditory nerve fibers directly, leading to the perception of sound in the brain and at least partial restoration of hearing function. The common denominator in most of these cochlear prosthesis systems has been the implantation into the cochlea of electrodes which are responsive to a suitable external source of electrical stimuli and which are intended to transmit those stimuli to the ganglion cells and thereby to the auditory nerve fibers.

A cochlear prosthesis operates by direct electrical stimulation of the auditory nerve cells, bypassing the defective cochlear hair cells that normally transduce acoustic energy into electrical activity in such nerve cells. In addition to stimulating the nerve cells, the electronic circuitry and the electrode array of the cochlear prosthesis performs the function of the separating the acoustic signal into a number of parallel channels of information, each representing the intensity of a narrow band of frequencies within the acoustic spectrum. Ideally, each channel of information would be conveyed selectively to the subset of auditory nerve cells that normally transmitted information about that frequency band to the brain. Those nerve cells are arranged in an orderly tonotopic sequence, from high frequencies at the basal end of the cochlear spiral to progressively lower frequencies towards the apex. In practice, this goal tends to be difficult to realize because of the anatomy of the cochlea.

Over the past several years, a consensus has generally emerged that the scala tympani, one of the three parallel ducts that make up the spiral-shaped cochlea, provides the best location for implantation of an electrode array used with a cochlear prosthesis. The electrode array to be implanted in this site typically consists of a thin, elongated, flexible carrier containing several longitudinally disposed and separately connected stimulating electrode contacts, perhaps 6-30 in number. Such electrode array is inserted into the scala tympani duct to a depth of about 10-30 mm via a surgical opening made at the basal end of the duct. During use, electrical current is passed into the fluids and tissues immediately surrounding the individual electrode contacts in order to create transient potential gradients that, if sufficiently strong, cause the nearby auditory nerve fibers to generate action potentials. The auditory nerve fibers arise from cell bodies located in the spiral ganglion, which lies in the bone, or modiolus, adjacent to the scala tympani on the inside wall of its spiral course. Because the density of electrical current flowing through volume conductors such as tissues and fluids tends to be highest near the electrode contact that is the source of such current, stimulation at one contact site tends to activate selectively those spiral ganglion cells and their auditory nerve fibers that are closest to that contact site. Thus, there is a need for the electrode contacts to be positioned close to the ganglion cells. This means, in practice, that the individual electrodes of the electrode array should be positioned on or near that surface of the electrode array closest to the modiolar wall.

Variously shaped cochlear electrode arrays are known in the art, including straight or slightly curved for lateral positioning, spiral shaped to generally conform to the shape of the scala tympani, and tightly spiral shaped to hug the modiolar wall. Regardless of which shape is used, the distal end of the electrode array must enter the cochleostomy in a straight configuration, and must be stiff enough to be pushed into the scala tympani. A modiolar hugging electrode array is typically straightened using a metal stylet inserted within a lumen of the electrode array; however, this presents several limitations.

FIGS. 1A-1C illustrate performance limitations of typical metal stylets used for implanting cochlear electrode arrays. FIG. 1A is a schematic diagram of an electrode array insertion, illustrating over insertion of the stylet, using a metal stylet 20 within stylet lumen 40 to straighten and stiffen an electrode array 70 for insertion into the cochlea 10, as is known in the art. Over insertion occurs when the stylet within the lead is allowed to enter too deep into the cochlea, such as deeper than 4 mm. This can cause the stiffened lead to press against the cochlea, and can also cause mismatch Between the electrode's curvature and the curvature of the cochlea as the electrode is inserted, leading to damage 22 of the delicate structures of the spiraling cochlea 10, including the basilar membrane and the spiral lamina. Therefore, during the implantation surgery, the surgeon must push the electrode array 70 off the metal stylet 20, which is generally being held with forceps, and into the cochlea 10. This is known as an “off stylet technique,” which can be difficult because of the small space and limited visibility. Note that only portion 12 above footpad 14 is visible to the implanting surgeon, who must try to determine the optimum insertion depth 18 to which the metal stylet-supported electrode array 70 should be inserted, at which point he must then continue to push the electrode array deeper into the cochlea, without continuing to push the metal stylet 20 any deeper, in order to avoid damage 22.

Because metal stylets are only partially inserted into the cochlea there is the possibility of over- and under-insertion. For cochlear electrode arrays, metal stylets are used to hold the electrode array straight for insertions, and these pre-curved electrode arrays are shaped to match the geometry of the cochlea. Advancing an electrode off of a stylet that has been over- or under-inserted into the cochlea 10 changes the relationship between the geometry of the electrode and that of the cochlea. For example, as depicted in FIG. 1B, overinsertion of the stylet may lead to a more lateral wall positioning or “buckling” of the unsupported electrode, particularly for leads that are not pre-curved. Underinsertion can lead to the tip of the electrode contacting the modiolus and folding up on itself, termed “pile up” 28, as depicted in FIG. 1C.

Another problem with metal stylets is the lack of interchangeability with current insertion tools. For example, various electrode array configurations, such as a tightly curled and banana-shaped, require different insertion tools that are not interchangeable, limiting the ability of the surgeon to select equipment based on his preferences and on particular patient needs.

Thus, while it has long been thought that proper atraumatic placement of the electrode array within the cochlea, using an electrode array that is atraumatic in long-term implant, would enhance performance of a cochlear implant, designers have faced problems in attempting to achieve these goals.

### SUMMARY OF THE INVENTION

The present invention addresses the above and other needs by providing a composite stylet that is relatively stiff and straight when outside the body to facilitate implantation of a compliant electrode array into a left or right cochlea, wherein the distal portion becomes relatively compliant at body temperature during insertion.

### BRIEF DESCRIPTION OF DRAWINGS

The above and other features and advantages of the present invention will be more apparent from the following more particular description thereof, presented in conjunction with the following drawings wherein:

FIGS. 1A-1C depict the limitations of a metal stylet;

FIG. 2 illustrates a composite stylet within an electrode array in the cochlea in accordance with the present invention;

FIG. 3 is a graph showing the general relationship between stiffness and temperature for the composite stylet;

FIG. 4 shows stiffness as a function of distance along the length of the composite stylet;

FIGS. 5A and 5B show the effect of shape on deflectability of the composite stylet; and

FIG. 6 is a side view of the composite stylet of the present invention as used in an exemplary electrode array, illustrating one preferred shape of the electrode array with the composite stylet inserted, both straight outside the body and curved within the cochlea (not shown).

Corresponding reference characters indicate corresponding components throughout the several views of the drawings.

### DETAILED DESCRIPTION OF INVENTION

The present invention addresses the above and other needs by providing a composite stylet having a glass transition temperature (T_{g}) between room temperature and body temperature, as will be described in more detail below, and a method for using the composite stylet to facilitate implantation of a compliant electrode array into a left or right cochlea. The following description is of the best mode presently contemplated for carrying out the invention. This description is not to be taken in a limiting sense, but is made merely for the purpose of describing the general principles of the invention. The scope of the invention should be determined with reference to the claims.

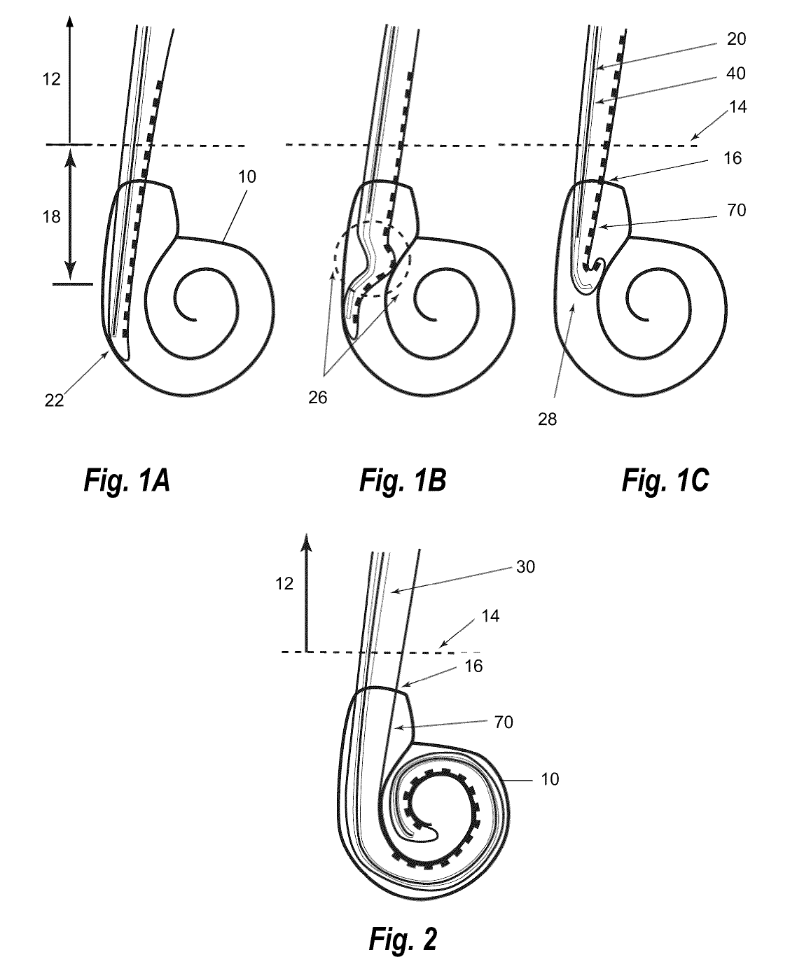

FIG. 2 is a schematic diagram showing a composite stylet 30 of the present invention within a lumen of electrode array 70 used to facilitate insertion of the electrode array into a cochlea 10. The electrode array 70 may have any shape known in the art suitable for cochlear implant, such as straight, slightly curved, spiral, or tightly spiraled, when unsupported by a stylet, and is described in further detail below. The electrode array 70 has a lumen that receives the composite stylet 30 to stiffen and straighten the electrode array for implant. The composite stylet 30 is fully inserted into the electrode array 70 and remains inserted throughout insertion of the electrode array into the cochlea 10. Note that this is markedly different from the insertion shown in FIG. 1A-C, in which the metal stylet 20 is held stationary as the electrode array 70 is pushed into the cochlea 10. Advantageously, the electrode array 70 stiffened with the composite stylet 30 can be safely inserted into the cochlea 10 to its final depth, thereby reducing the number of components that the surgeon must control to perform the insertion. Because the electrode array 70 can be inserted into the cochlea 10 without simultaneously removing the composite stylet 30 from the electrode array, the forces felt by the surgeon during implantation are the forces exerted on the electrode array by the cochlea. In contrast, as described above, a typical implant requires the surgeon to push the electrode array off a metal stylet, in which case what is felt is the combination of the force required to push the electrode array off of the metal stylet and the force exerted on the electrode array by the cochlea.

A composite material is a material comprising two or more phases and can have a variety of morphologies. Often, composites consist of a matrix phase and a dispersed phase and the properties of the composite are different from those of the constituents. The matrix phase usually has a continuous character and is more compliant and ductile than the dispersed phase which is imbedded in the matrix and is usually stiffer and stronger than the matrix. Besides strength, composites can be used to provide other benefits, such as stiffness, toughness, lower weight, improved processing characteristics, and lower cost. The composite stylet 30 comprises a composite material having a matrix phase, which, in some embodiments, comprises a thermoset or thermoplastic polymer; in some embodiments, the matrix phase may comprise an epoxy. The composite stylet 30 also comprises a reinforcing phase, which, in some embodiments, comprises glass or carbon fibers oriented in the direction of the stylet axis. Other fibers, such as Kevlar or metal can be used, and in some embodiments, these fibers may have different aspect ratios and are not necessarily oriented in the direction of the stylet axis, but may be randomly oriented. Furthermore, in some embodiments, the reinforcing phase is not necessarily fibers; other particulates can be used, such as flakes or other shapes.

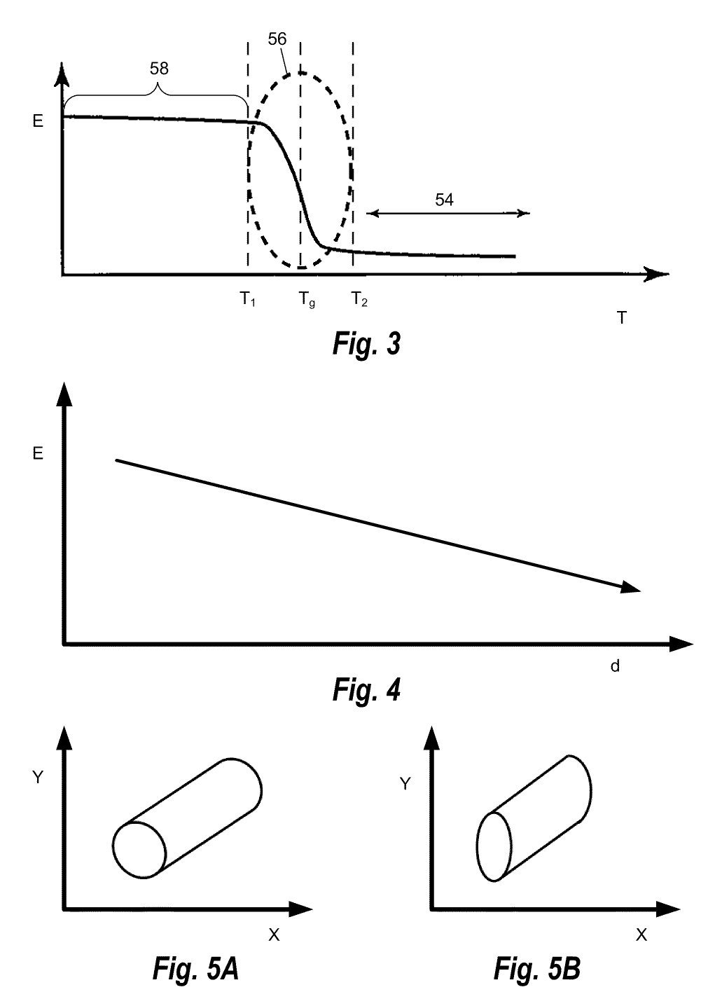

FIG. 3 is a graph showing the general relationship between stiffness and temperature for the composite stylet. The composite stylet 30 of the present invention is made from a composite material having at least one component that has a T_{g} within a glass transition region 56 between T₁ and T₂, wherein T₁ is less than T₂, and wherein T₁ may be about room temperature, such as between 15° C. and 35° C., between 20° C. and 30° C., or about 24° C., and T₂ may be body temperature or slightly below, such as between 17° C. and 37° C., between 24° C. and 36° C., or about 35° C., providing a difference of at least a factor of 2 in elastic modulus (E) above and below T_{g}. Below the T_{g}, in the glassy region 58, the composite stylet 30 is relatively stiff, having an elastic modulus of between 45 MPa and 340 GPa, or between 0.120 GPa and 340 GPa, or between 1 GPa and 340 GPa, or between 20 GPa and 140 GPa, for example, approximately 85 GPa. Above the T_{g}, in the rubbery region 54, the composite stylet 30 is relatively compliant with the elastic modulus reduced by at least a factor of 2. For example, in one embodiment, if the room temperature elastic modulus is 20 GPa or more, the elastic modulus at body temperature can be 10 GPa or less. In some embodiments, the body temperature elastic modulus is reduced to as low as possible, such as between 0.02 GPa and 1.4 GPa, depending on the room temperature modulus, to ensure a reduction in elastic modulus of at least a factor of 2. Thus, as the electrode array 70 is inserted into the cochlea 10, and as the composite stylet 30 carried therein is warmed above room temperature, the composite stylet 30 softens such that the electrode array 70 is no longer straightened by the composite stylet 30. This provides the implanting surgeon with the desired combination of stiffness outside the cochleostomy to facilitate pushing the electrode array 70 into the cochlea without buckling, and compliance inside the cochlea to enable the electrode array to turn and reduce insertion forces, and to reduce residual forces on the cochlea during long term implant.

Generally, thermosets have a wider glass transition region 56 than thermoplastics, with thermoplastics having a more sharply defined T_{g}; i.e., as the material changes from room temperature to body temperature, the change in stiffness is more gradual for a thermoset than for a thermoplastic. The T_{g} of a material may be defined as a single value, e.g., 27° C., which is somewhat arbitrary and the T_{g} may be better expressed as the range of temperatures over which the glass transition occurs, such as 24° C.-34° C. Selection of thermoset or thermoplastic for the matrix material, then, will depend on whether a gradual or sharp change in stiffness is desired, and depending on ability to tune the glass transition temperature and ease of fabrication.

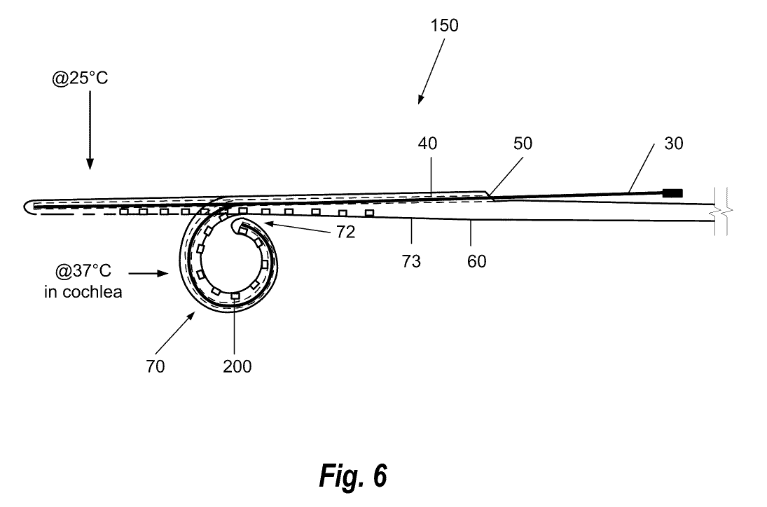

FIG. 4 shows elastic modulus as a function of distance along the length of one embodiment of composite stylet 30 having a high modulus at the portion that remains outside the body, gradually decreasing modulus toward the stylet portion that will be inserted into the basal region of the cochlea, and further decreasing in modulus toward the distal tip 72 (FIG. 6). This graded modulus may be accomplished by incorporating the reinforcing phase, such as carbon or glass fibers, in a nonuniform distribution along the stylet length as needed for atraumatic insertion. The shape of this plot is just an example and many other shapes are possible. This graded modulus may be monotonically decreasing from the proximal to distal end of the lead, such as linearly, as shown; alternatively, it may decrease nonlinearly, such as by steps, or may have various stiffer regions to serve any particular purpose. Alternatively or additionally, the T_{g} may be varied along the length of the composite stylet 30 to provide graded stiffness to facilitate insertion. The T_{g} of the tip portion may be selected such that the very end of the stylet tip is soft both above and below room temperature, while the portion of the stylet that resides in the electrode array portion at the basal end of the cochlea may be such that it does not soften at body temperature.

Optionally, the matrix material may comprise a shape memory thermoset or thermoplastic polymer. Such shape memory polymers are known in the art, such as those described, for example, in U.S. Publication 2007/0073130, incorporated herein by reference, and may be used to preform the stylet to have two different unconstrained shapes, depending on temperature. The stylet may be constructed to assume a substantially straight configuration when unconstrained at room temperature, and to take on a slightly curved or spiral shape when unconstrained, such as by the electrode array and/or cochlea, at or slightly below body temperature. This property of the stylet having a curved shape at body temperature allows the electrode array to better conform to the cochlea to improve the positioning inside the cochlea and reduce electrode insertion forces. An example of a cochlear electrode array with a positioning stylet comprising a memory wire is taught in U.S. Pat. No. 6,119,044 to Kuzma, incorporated herein by reference, the teachings of which can be applied to the composite stylet of the present invention. Unlike metallic shape-memory stylets, which exhibit only a small change in modulus with temperature, a polymer-based composite shape-memory stylet can be designed to undergo a dramatic modulus change as it passes through the glass transition temperature, even while having a preferred curved shape in its compliant state. Furthermore, it should be noted that the glass transition temperature is not necessarily the same temperature as the shape transformation temperature. For example, if the T_{g} is less than the shape transformation temperature, as the stylet begins to increase in temperature by insertion into the body, it will first begin to decrease elastic modulus and then start to preferentially assume a curved shape. On the other hand, if T_{g} is greater than the shape transformation temperature, upon initiation of insertion, the stylet will first begin to take on a curved shape, and will subsequently begin to soften. The shape memory properties of the composite stylet 30 may be utilized to help assure that the electrode contacts of the electrode array 70 will be optimally positioned facing the modiolar wall and to prevent damage to the delicate structures of the cochlea.

FIGS. 5A and 5B show cross sectional shapes and indicate the effect of the shape of the composite stylet on the asymmetry of the stiffness. In FIG. 5A, the stylet has a circular cross section and is therefore is equally easy to deflect in any direction. The stylet shown in FIG. 5B is elliptical in cross section, and is easily deflectable in the X direction but is resistant to deflection in the Y direction. The resulting anisotropic stiffness of the electrode array may be utilized to guide the electrode array as needed into the cochlea to reduce trauma. For example, the composite stylet may be shaped to turn easily and resist vertical deflection which can lead to rupturing of the basilar membrane. The composite stylet 30 may be manufactured in a variety of shapes to ensure that the stiffness is optimized in the different directions of the electrode array. As examples, the composite stylet may have a cross-section that is rectangular, ellipsoidal, circular, or square.

The composite stylet 30 of the present invention is used with a electrode array 70 that has a stylet lumen 40 and may be of a variety of shapes and constructions. For example, in its relaxed condition outside the body, unsupported by a stylet and unconstrained by the cochlea, the electrode array 70 may be straight, such as that described in U.S. Pat. Nos. 6,757,970 and 7,047,081; curved, as described in U.S. Pat. No. 7,315,763; or spiral shaped, such as described in U.S. Pat. Nos. 6,604,283; 6,125,302; 7,319,906; and U.S. Publication 2008/0027527, all of which are incorporated herein by reference. The composite stylet 30 of the present invention may be used with the electrode array as described in a co-pending Provisional application titled “Cochlear Electrode Array” to Timothy Beerling et al., attorney docket number 08-00012-01, filed on May 22, 2009, which is incorporated herein by reference.

FIG. 6 shows an exemplary electrode array 70 used with the inventive composite stylet 30. As mentioned above, the electrode array 70 may be straight, curved, or spiral when unsupported by a stylet outside the body, but must be substantially straight for insertion into the cochlea, and takes on the spiral shape when implanted in the cochlea. FIG. 6 shows the electrode array 70 in two configurations, both having the composite stylet 30 inserted, outside the body at room temperature and as implanted in the cochlea (cochlea not shown) at body temperature. The electrode array 70 forms the distal end of a cochlear lead 150 adapted to be connected to an implantable cochlear stimulator (not shown), which is typically housed within a case having an array of feedthrough terminals corresponding to its multiple channels. The electrode array 70 includes a flexible carrier body 60 having a lumen 40 that passes longitudinally therethrough. A multiplicity of spaced-apart electrode contacts 200 are formed on the carrier body. The flexible carrier body 60 may be molded from silicone rubber or any other suitable flexible material as is known in the art, and is adapted to assume a spiral shape when implanted within the cochlea. The electrode contacts 200 are spaced apart along a medial side 73 of the carrier body 60, which is that side on the inside curve of the spiral when implanted, to face the modiolar wall, close to the ganglion cells to be stimulated. These electrode contacts 200 are respectively connected to the wire conductors (not shown) within the lead 150. The electrode array 70 may further include one or more reference markers (not shown) to provide insertion depth information to the implanting surgeon.

The lumen 40 passes longitudinally through the flexible carrier body 60 up to a distal tip 72, where the lumen 40 is closed. The lumen 40 has a diameter sufficiently large to allow a composite stylet 30 to be slidably inserted therein from the stylet lumen entrance 50. To enable the electrode array 70 to be straightened and thereafter curved or spiraled, the composite stylet 30 is dimensioned slightly smaller than the lumen 40 to be free to slide within the lumen 40 to prevent binding. Small changes in stylet diameter have a large impact on its stiffness. While stiffness is directly proportional to the elastic modulus, stiffness varies as the fourth power of the stylet diameter. In some embodiments, the composite stylet 30 has a diameter of between 0.003 and 0.018 inches, or between 0.004 and 0.018 inches, or between 0.004 and 0.009 inches, or about 0.006 to 0.007 inches. The stylet may have a constant diameter or may vary monotonically along its length, such as tapering to a smaller diameter at the distal end. For example, the stylet may have a diameter of about 0.012 inches along most of its length, beginning to taper 14 to 40 mm from the distal end, tapering for a length of 8 to 10 mm down to a diameter of about 0.006 inches 6 to 10 mm from the distal end, and remaining a substantially constant diameter for the most distal 6 to 10 mm. There may be multiple tapered steps along the length of the stylet or the stylet may taper continuously. Additionally or alternatively, the stylet may vary in shape along its length, for example being round at the proximal end and transitioning to elliptical or flat at the distal end. Furthermore, this cross sectional shape change may be abrupt, or in steps, or continuously.

In one embodiment, the lead is packaged with the stylet inserted in the lead. A protective sleeve or other protective packaging is used to keep the lead with stylet in a straight configuration to avoid the stylet taking a curved set, which is especially important in case the device were exposed to elevated temperatures during shipping. Packaging the lead with the stylet already preloaded allows it to be conveniently implanted without having to take the step of inserting the stylet. Alternatively, the stylet may be packaged separately and inserted in the operating room. In case the stylet needs to be loaded or reloaded into the lead, the reloading tool taught in U.S. Publication 2008/0109011, which is hereby incorporated by reference, may be used to load the composite stylet 30 into the electrode array 70.

Whether prepackaged with the stylet inserted or inserted in the operating room, prior to implant, the composite stylet 30 is slidably inserted into the lumen 40 until the tip of the composite stylet 30 reaches the distal tip 72 of the electrode array 70 to stiffen it to approximately straight or slightly curved for insertion into the cochlea. The electrode array 70 is then inserted into the cochlea. As the composite stylet 30 warms to body temperature, at least a portion of it passes through its T_{g}, becoming more compliant, allowing the electrode array 70 to conform to the spiral cochlea.

Advantageously, the structure of the electrode array 70 itself facilitates bending of the array in the medial direction with the electrode contacts 200 on the inside of the bend, yet deters lateral flexing or twisting of the array that would tend to position or point the electrode contacts away from the modiolar wall. Thus, the electrode array 70 is able to follow the scala tympani duct as it is inserted deeper into the cochlea while electrode contacts 200 remain facing the modiolar wall. When the electrode array 70 with composite stylet 30 is inserted into the scala tympani duct of a cochlea, the composite stylet warms and softens, allowing the array to assume a spiral shape with the electrode contacts 200 on the medial side 73 of the electrode array 70 facing the modiolar wall. Additionally or alternatively, as described above, the composite stylet 30 may be constructed to be stiffer out of plane of the electrode array than in the medial direction, suppressing deflection of the electrode towards the basilar membrane, scala media, osseous spiral lamina (OSL), and spiral ligament. In any case, because the electrode contacts of the electrode array remain facing the modiolar wall, stimulation of the cells within the modiolar wall may occur at lower energy than would be required if they were not facing the modiolar wall.

To further enhance the performance of the composite stylet a coating may be applied to reduce the frictional forces encountered during insertion into the electrode lumen. Alternative approaches, such as surface modification of the composite stylet, may also be used to reduce these frictional forces.

The present invention provides a composite stylet that is easy to manufacture using low cost technology. The stylets may be molded, such as by resin transfer molding, injection molding, or compression molding. Alternatively, they may be extruded or made by a die pulling process such as pultrusion. The cross sectional shape and size may be varied along the length directly using a molding process or by a variable diameter extrusion process. Alternatively, a drawing process or centerless grinding may be used to produce radially symmetrical tapers. To create cross sectional shape changes, such as from circular to flat or elliptical, thermoplastics may be postformed using heat and pressure. Other shape or dimensional variations can be made by secondary machining operations.

In an alternative embodiment, the composite stylet is adapted to permanently reside within a lumen of the carrier body of the electrode array. The lead may be prepackaged with the composite stylet already inserted, or the stylet may be inserted into the lead just before implant. Before implant, the entire array with embedded stylet is straight when at room temperature to facilitate electrode insertion into the cochlea. During electrode array insertion, the stylet warms to body temperature and softens, allowing the electrode array to take on a spiral shape, either by returning to a relaxed spiral condition (in the case of a spiral-shaped array) or by becoming compliant enough to conform to the spiral shape of the cochlea (for a straight or slightly curved array). The stylet may include a knob for ease of insertion, which is designed to be broken or cut off or otherwise removed from the stylet once the lead is fully inserted.

Because the composite stylet of the present invention can remain in the electrode array as it is inserted into the cochlea, it is possible to use other insertion tools concurrently, providing surgeons with an expanded array of options for how they insert devices and thereby improve ease of use and patient outcomes. For example, the lead insertion tool described in U.S. Provisional Application 61/046,302, which is herein incorporated by reference, may be used with the composite stylet of the present invention to implant the lead into the cochlea. The composite stylet 30 reduces complexity from the implantation process and provides greater flexibility for the surgeons by allowing many different electrode insertion method options.

It is another aspect of the present invention to provide a kit containing multiple stylets having a range of properties tuned to the various conditions anticipated. Various kits may be provided based on the type of procedure being performed, e.g., a set of stiffer stylets for revisions or procedures in which the patients have significant ossification. The lead may be packaged with the most commonly-used stylet already inserted, with other stylets available for insertion using a stylet loading tool.

The composite stylet 30 of the present invention provides improved ease of insertion for the surgeon, increased insertion depth, decreased trauma, and improved placement within the cochlea, reducing the risk of trauma and damage to residual hearing. The stylet stiffness changes during the insertion process, providing the optimum stiffness and compliance at the appropriate point during the procedure, whereby stiffness prevents buckling and allows the surgeon to push the device into the cochlea, and compliance inside of the cochlea reduces trauma. This technology can improve insertion by having a preformed shape to enhance the positioning of the electrode array inside the cochlea and/or reduce insertion forces, improving patient outcomes.

While the invention herein disclosed has been described by means of specific embodiments and applications thereof, numerous modifications and variations could be made thereto by those skilled in the art without departing from the scope of the invention set forth in the claims. Furthermore, although a composite stylet and method for use to implant a cochlear electrode array into the cochlea has been described, the principles of the invention may be applied to other types of implantable leads for applications other than cochlear stimulation.

1. A cochlear electrode stylet having a proximal end and a distal end, comprising:
an elongated member comprising a composite of a polymer and a reinforcing material, wherein upon insertion of the distal end into a cochlea, a portion of the stylet within the body softens to reduce the elastic modulus of the portion by at least a factor of 2 in response to cochlear temperature.

2. The stylet of claim 1, wherein the elastic modulus at room temperature is at least 1 GPa.

3. The stylet of claim 1, wherein the diameter of the portion is between 0.004 and 0.018 inches.

4. The stylet of claim 1, wherein the portion softens prior to reaching its final implant depth in the cochlea.

5. The stylet of claim 1, wherein the reinforcing material is not uniformly distributed along the stylet such that at a given temperature, the stylet has a higher elastic modulus at a basal portion than at a distal portion.

6. The stylet of claim 1, wherein the reinforcing material comprises glass or carbon fibers.

7. The stylet of claim 1, wherein the stylet has a noncircular cross section.

8. The stylet of claim 1, wherein the room temperature elastic modulus varies along its length.

9. The stylet of claim 1, wherein the polymer comprises a polyurethane.

10. The stylet of claim 1, wherein the polymer comprises an epoxy.

11. The stylet of claim 1, wherein the polymer comprises a shape memory material and wherein the stylet assumes a substantially straight unconstrained configuration at room temperature and assumes a curved unconstrained configuration at body temperature.

12. A method of implanting a cochlear electrode array, comprising:
inserting a stylet into a lumen of the cochlear electrode array such that a distal end of the stylet is substantially at a distal end of the electrode array, the stylet comprising a composite of at least one polymer and at least one reinforcing material, the composite having a glass transition temperature of between about room temperature and about body temperature; and
inserting the distal end of the electrode array through a cochleostomy while maintaining the stylet distal end substantially at the electrode array distal end and simultaneously allowing the portion of the stylet within the electrode array to soften as it is heated by the body while the portion of the stylet outside the electrode array remains below the glass transition temperature.

13. The method of claim 12, wherein a portion of the stylet softens to reduce the elastic modulus by at least a factor of 2 in response to cochlear temperature.

14. The method of claim 12, further including:
removing the stylet from the electrode array.

15. The method of claim 12, further including:
allowing the stylet to permanently reside in the electrode array.

16. The method of claim 12, wherein the electrode array has a relaxed shape of a spiral at room temperature when not inside the cochlea and when not stiffened with a stylet.

17. The method of claim 12, further comprising the step of utilizing an additional insertion tool while the stylet is in the lumen.

18. A cochlear electrode array implant stylet kit, comprising:
a first stylet comprising a composite of a polymer and a reinforcing material, wherein upon insertion of a distal end of the first stylet into a cochlea, a portion of the first stylet within the body softens to reduce the elastic modulus by at least a factor of 2 in response to cochlear temperature; and
a second stylet comprising a composite of a polymer and a reinforcing material, wherein upon insertion of a distal end of the stylet into a cochlea, a portion of the second stylet within the body softens to reduce the elastic modulus by at least a factor of 2 in response to cochlear temperature, wherein
the second stylet has at least one property different from the properties of the first stylet.

19. The kit of claim 18, wherein the portion of the first stylet has an elastic modulus at room temperature of at least 1 GPa.

20. The kit of claim 18, wherein the first stylet reinforcing material comprises a first loading fraction of fibers and wherein the second stylet reinforcing material comprises a second loading fraction of fibers different from the first loading fraction of fibers such that the at least one property different from the properties of the first stylet is at least one of room temperature elastic modulus and body temperature elastic modulus.

21. The kit of claim 18, wherein the second stylet composite has a different glass transition temperature than the first stylet composite.
